(19) United States
(12) Patent Application Publication
(10) Pub. No.: US 2026/0215832 A1
(43) Pub. Date: Jul. 30, 2026

(54) Methods, Systems, and Devices for Making One or More Surgical Guides

(71) Applicant: Vanduzen, Inc. d/b/a MedCAD, Dallas, TX (US)

(72) Inventor: Nancy Yvonne Hairston, Dallas, TX (US)

(21) Appl. No.: 19/455,453

(22) Filed: Jan. 21, 2026

Related U.S. Application Data
(60) Provisional application No. 63/750,078, filed on Jan. 27, 2025.

(51) Int. Cl. A61B 17/90; A61B 17/00; A61B 34/10; B23C 3/16

(52) U.S. Cl. CPC A61B 17/90; A61B 34/10; B23C 3/16; A61B 2017/00526; A61B 2034/102; A61B 2034/105

(57) ABSTRACT

Provided herein are methods, system, and devices for method of making one or more surgical guides and implants comprising: obtaining or having obtained one or more three-dimensional (3-D) image file(s) of an ankle, a foot, or both; generating a surgical plan and hardware dataset; using the surgical plan and hardware dataset to 3-D print or mill one or more surgical guides: opening(s) for inserting a bone screw(s) or pin(s); slots for drilling or cutting bone; slot(s) along an edge of the surgical guide(s) for aligning the surgical guide(s) for receiving a bone screw or pin; a surface that is contoured to match a surface of a target bone; indicia or predictive hole feature(s) for marking or aligning the surgical guide(s) to a bone or for marking a designation for the guide(s).

### CROSS-REFERENCE TO RELATED APPLICATIONS

This application claims priority to U.S. Provisional Application Ser. No. 63/750,078 filed Jan. 27, 2025, the entire contents of which are incorporated herein by reference.

### TECHNICAL FIELD

The present disclosure relates in general to the field of surgical plans and guides, and more particularly, to novel methods, system, and devices for making one or more surgical guides.

### STATEMENT OF FEDERALLY FUNDED RESEARCH

None.

### BACKGROUND

Without limiting the scope of the disclosure, its background is described in connection with foot and ankle surgery guides.

One such device and method is taught by Hafez and Al Nehayan in U.S. Patent Publication No. 20230310051, entitled “Patient Specific Instrument (PSI) Method & Device for Percutaneous Fixation of Fractures”. These applicants are said to teach a patient-specific surgical guide for percutaneous fixation of fractures designed for detection of the point of insertion, direction and angle of metal-ware (wires, screws, plates or nails). The patient-specific surgical guide is said to be seated on a bony landmark and the interior surface of the guide matches the surface anatomy of bones and fit in a single secure position for the reduction of bony or joint displacement and to insert wires or metal ware in bone and joints without need for fluoroscopic imaging.

Another such system is taught by Campagnoli and Keuper, in U.S. Patent Publication No. 20210228221, entitled “Self-Positioning Drill Guide”. These applicants are said to teach surgical constructs, assemblies and methods of tissue fixation are disclosed. A self-positioning drill guide is said to automatically reference a drill point or drill angle for drilling tunnels in bones by using peripheral edges. The self-positioning drill guide is further said to include a center point drill guide configured to automatically center drill tunnels in bones by using peripheral edges that precisely reference the center point. It is said that the centering guide helps to automatically determine the center of bones as well as to provide increased control during drilling.

Finally, another such system is taught by Hafez and Ahmed in in U.S. Patent Publication No. 20200367910, entitled “A Three-In-One Patient-Specific Template For Usage In Ankle Replacements Surgeries”. These applicants are said to teach a custom-made cutting block for total ankle replacement consisting of two pieces, one for tibia and other for talus. It is said that the block can be used in 3 ways as: pin locator, cutting block or in coupling with the other conventional instruments. Finally, the block is said to provide a unique method for matching with the tibia and talus based on the CT-scan imaging.

### Orthopedic Osteotomy Precision Studies Comparing Current State of Art to Guided Surgery.

Viehöfer, et al., reported that in 1st MET Cuts, that there were no advantages noted from the use of augmented reality (AR) advantage noted for experienced surgeons, while with inexperienced: 6.4+/−3.5 deg compared with freehand 10.5+/−5.5 deg. Viehöfer, A. F., Wirth, S. H., Zimmermann, S. M. et al. Augmented reality guided osteotomy in hallux Valgus correction. BMC Musculoskelet Disord 21, 438 (2020). doi.org/10.1186/s12891-020-03373-4.

Next, Berlet, et al, reported a placement from the preoperative plan was less than 2 degrees in all angular DOF, providing greater accuracy than the +3 degrees determined in other implant system studies using traditional instrumentation and computer navigation. Berlet, G. C., Penner, M. J., Lancianese, S., Stemniski, P. M., & Obert, R. M. (2014). Total Ankle Arthroplasty Accuracy and Reproducibility Using Preoperative CT Scan-Derived, Patient-Specific Guides. Foot & ankle international, 35(7), 665-676. doi.org/10.1177/1071100714531232.

Finally, Hetherington, et al., reported that made freehand, the average angle was 71.9. for the fourth-year student, 55.5° for the third-year resident, and 70.7° for the podiatric physician. For the osteotomies made using only a guide wire, the average angle was 76.1° for the fourth-year student, 58.0° for the third-year resident, and 69.9° for the podiatric physician. The results of this study clearly demonstrate that using an osteotomy guide provides greater accuracy of the apical angle of the osteotomy and inherent stability of the osteotomy, despite the experience level of the performing individual. Hetherington, V. J., Kawalec-Carroll, J. S., Melillo-Kroleski, J., Jones, T., Melillo, M., McFarland, N., Blazer, M., & Favazzo, J. A. (2008). Evaluation of surgical experience and the use of an osteotomy guide on the apical angle of an Austin osteotomy. Foot (Edinburgh, Scotland), 18(3), 159-164. doi.org/10.1016/j.foot.2008.01.008.

Despite these advances, a need remains for customized guides, instruments, models, and/or inserts that are customized for a specific patient. A further need remains for an intuitive system that surgeons can use to plan surgeries of the foot and/or ankle, surgical plans, surgical models, surgical guides, and surgical inserts for patient-specific use.

### SUMMARY

As embodied and broadly described herein, an aspect of the present disclosure relates to a method of making one or more surgical guides and implants comprising: obtaining or having obtained one or more three-dimensional (3-D) image file(s) of an ankle, a foot, or both with a medical imaging system; providing an interactive surgery planning tool that generates a surgical plan and hardware dataset comprising one or more steps in a surgical procedure of the 3-D image, one or more surgical guides, one or more implants, or both the surgical guides and implants; using the surgical dataset to generate a surgical guide dataset for one or more surgical guides, one or more surgical implants, or both; and using the surgical guide dataset to at least one of: 3-D printing or milling one or more surgical guides using the surgical guide dataset, the one or more surgical guides comprising at least one of: one or more openings for inserting a bone screw or pin; one or more slots for drilling or cutting bone; one or more slots along an edge of the one or more surgical guides for aligning the one or more surgical guides for receiving a bone screw or pin; a surface that is contoured to match a surface of a target bone; one or more indicia or predictive hole features for marking or aligning the one or more surgical guides to a bone or for marking a designation for one or more guides. In one aspect, the method further comprises 3-D printing or milling one or more surgical inserts using the surgical guide dataset. In another aspect, the method further comprises providing a step-by-step guide for the use of the one or more surgical guides, the one or more surgical inserts, or both. In another aspect, the step-by-step guide is printed, electronic, or displayed as a 3-D rendering. In another aspect, the one or more images comprise at least one of: one or more X-rays, one or more computed tomography (CT) scans, one or more cone beam computed tomography (CBCT) scans, one or more magnetic resonance imaging (MRI) scans, an ultrasound scan, or one or more positron emission tomography (PET) scanning. In another aspect, the one or more surgical guides, the one or more surgical inserts, or both comprise at least one of: titanium, titanium alloy, stainless steel, stainless steel alloy, aluminum, aluminum alloy, carbon fiber composite, polymeric, ceramic, composite materials, biocompatible polymer matrix reinforced with bioactive fillers and/or fibers, polyetherether-ketone (PEEK), polyvinyl chloride (PVC), polyethylene polyesters, polycarbonate, Teflon coated metal, ultra high molecular weight polyethylene (UHMWPE), urethane di methacrylate (DUDMA)/tri-ethylene glycol dimethacrylate (TEDGMA) blended resins with fillers and/or fibers nitinol, polyphenylsulfone, polycarbonate, polyetherimide, polyetherketoneketone (PEKK), polyaryletherketone (PAEK), acrylic, cobalt chrome, polyamide, or acrylonitrile butadiene styrene (ABS). In another aspect, the one or more surgical guides, the one or more surgical inserts, or both comprise at least one of: bioabsorbable polymers, bioresorbable polymers, bone powder, growth factors, analgesics, antibiotics, steroids, immunosuppressants, immunostimulators, anti-inflammatory agents, osteocalcin, osteoglycin and growth factors such as bone morphogenic proteins (BMP-2, -4, -6, -7 and -9), fibroblast growth factors (FGFs), vascular endothelial growth factors (VEGFs), platelet derived growth factors (PDGFs), and transforming growth factors, antifungal agents, and antiviral agents; silver or copper micro- or nano-particles, cartilage shavings, abrasion chondroplasty, s cartilage cell allografts, stem cell autografts, s perichondral autografts, periosteal autografts, cartilage scaffolds, osteoarticular autografts, or allografts. In another aspect, the one or more surgical guides, the one or more surgical inserts, or both are manufactured to treat bone trauma, bone fractures, cancer, juvenile hallux valgus, clubfoot, congenital vertical talus, calcaneovalgus deformity, metatarsus adductus, flexible flatfoot, tarsal coalitions, or cavus foot. In another aspect, the method further comprises 3-D printing one or more anatomical models of the one or more bones using the three-dimensional (3-D) image file of an ankle, a foot, or both. In another aspect, the interactive surgery planning tool can be used remotely to generate the one or more surgical guides, the one or more surgical inserts, or the one or more anatomical models of the one or more bones. In another aspect, the one or more surgical guides are configured to conform to a surface of the bones of the foot or ankle of a specific patient. In another aspect, the one or more surgical guides comprise one or more of: holes, slots, markers, pins, points, or indicia for aligning the surgical guide to a target location of the one or more bones or the foot or ankle. In another aspect, the one or more guides are used for high tibia osteotomy, proximal phalanx and metatarsal arthroplasty, lapidus (bunion), charcot (flat foot), complex and/or revision cases in the mid and fore foot, cavus (high arch) foot correction, calcaneus fractures, or talus replacement

As embodied and broadly described herein, an aspect of the present disclosure relates to a system for making one or more surgical guides and implants comprising: obtaining or having obtained one or more three-dimensional (3-D) image file of an ankle, a foot, or both with a medical imaging system; providing an interactive surgery planning tool that generates a surgical plan and hardware dataset comprising one or more steps in a surgical procedure of the 3-D image, one or more surgical guides, one or more implants, or both the surgical guides and implants; using the surgical dataset to generate a surgical guide dataset for one or more surgical guides, one or more surgical implants, or both; and one or more printers or mills that following the surgical guide dataset at least one of: 3-D printing or milling one or more surgical guides using the surgical guide dataset, the one or more surgical guides comprising at least one of: one or more openings for inserting a bone screw or pin; one or more slots for drilling or cutting bone; one or more slots along an edge of the one or more surgical guides for aligning the one or more surgical guides for receiving a bone screw or pin; a surface that is contoured to match a surface of a target bone; one or more indicia or predictive hole features for marking or aligning the one or more surgical guides to a bone or for marking a designation for one or more guides. In one aspect, the system further comprises 3-D printing or milling one or more surgical inserts using the surgical guide dataset. In another aspect, the system further comprises providing a step-by-step guide for the use of the one or more surgical guides, the one or more surgical inserts, or both. In another aspect, the step-by-step guide is printed, electronic, or displayed as a 3-D rendering. In another aspect, the one or more images comprise at least one of: one or more X-rays, one or more computed tomography (CT) scans, one or more cone beam computed tomography (CBCT) scans, one or more magnetic resonance imaging (MRI) scans, an ultrasound scan, or one or more positron emission tomography (PET) scanning. In another aspect, the one or more surgical guides, the one or more surgical inserts, or both comprise at least one of: titanium, titanium alloy, stainless steel, stainless steel alloy, aluminum, aluminum alloy, carbon fiber composite, polymeric, ceramic, composite materials, biocompatible polymer matrix reinforced with bioactive fillers and/or fibers, polyetherether-ketone (PEEK), polyvinyl chloride (PVC), polyethylene polyesters, polycarbonate, Teflon coated metal, ultra high molecular weight polyethylene (UHMWPE), urethane di methacrylate (DUDMA)/tri-ethylene glycol dimethacrylate (TEDGMA) blended resins with fillers and/or fibers nitinol, polyphenylsulfone, polycarbonate, polyetherimide, polyetherketoneketone (PEKK), polyaryletherketone (PAEK), acrylic, cobalt chrome, polyamide, or acrylonitrile butadiene styrene (ABS). In another aspect, the one or more surgical guides, the one or more surgical inserts, or both comprise at least one of: bioabsorbable polymers, bioresorbable polymers, bone powder, growth factors, analgesics, antibiotics, steroids, immunosuppressants, immunostimulators, anti-inflammatory agents, osteocalcin, osteoglycin and growth factors such as bone morphogenic proteins (BMP-2, -4, -6, -7 and -9), fibroblast growth factors (FGFs), vascular endothelial growth factors (VEGFs), platelet derived growth factors (PDGFs), and transforming growth factors, antifungal agents, and antiviral agents; silver or copper micro- or nano-particles, cartilage shavings, abrasion chondroplasty, s cartilage cell allografts, stem cell autografts, s perichondral autografts, periosteal autografts, cartilage scaffolds, osteoarticular autografts, or allografts. In another aspect, the one or more surgical guides, the one or more surgical inserts, or both are manufactured to treat bone trauma, bone fractures, cancer, juvenile hallux valgus, clubfoot, congenital vertical talus, calcaneovalgus deformity, metatarsus adductus, flexible flatfoot, tarsal coalitions, or cavus foot. In another aspect, the system further comprises 3-D printing one or more anatomical models of the one or more bones using the three-dimensional (3-D) image file of an ankle, a foot, or both. In another aspect, the interactive surgery planning tool can be used remotely to generate the one or more surgical guides, the one or more surgical inserts, or the one or more anatomical models of the one or more bones. In another aspect, the one or more surgical guides are configured to conform to a surface of the bones of the foot or ankle of a specific patient. In another aspect, the one or more surgical guides comprise one or more of: holes, slots, markers, pins, points, or indicia for aligning the surgical guide to a target location of the one or more bones or the foot or ankle.

As embodied and broadly described herein, an aspect of the present disclosure relates to a computer-readable storage medium comprising instructions stored thereon that, in response to execution by a processor, causes an apparatus to print or mill one or more surgical guides, one or more surgical implants, or both, comprising: obtaining, or having obtained, and storing one or more three-dimensional (3-D) image file of an ankle, a foot, or both with a medical imaging system; a processor comprising an interactive surgery planning tool that: generates a surgical plan and hardware dataset comprising one or more steps in a surgical procedure of the 3-D image, one or more surgical guides, one or more implants, or both the surgical guides and implants; using the surgical dataset to generate a surgical guide dataset for one or more surgical guides; and 3-D printing or milling one or more surgical guides using the surgical guide dataset, the one or more surgical guides comprising at least one of: one or more openings for inserting a bone screw or pin; one or more slots for drilling or cutting bone; one or more slots along an edge of the one or more surgical guides for aligning the one or more surgical guides for receiving a bone screw or pin; a surface that is contoured to match a surface of a target bone; one or more indicia or predictive hole features for marking or aligning the one or more surgical guides to a bone or for marking a designation for one or more guides. In one aspect, the computer-readable storage medium displays or prints a step-by-step guide for the use of the one or more surgical guides, the one or more surgical inserts, or both. In another aspect, the step-by-step guide is printed, electronic, or displayed as a 3-D rendering. In another aspect, the one or more images comprise at least one of: one or more X-rays, one or more computed tomography (CT) scans, one or more cone beam computed tomography (CBCT) scans, one or more magnetic resonance imaging (MRI) scans, an ultrasound scan, or one or more positron emission tomography (PET) scanning. In another aspect, the one or more surgical guides, the one or more surgical inserts, or both comprise at least one of: titanium, an alloy of titanium, nitinol, stainless steel, plastic, or poly ethyl ketone (PEEK). In another aspect, the one or more surgical guides, the one or more surgical inserts, or both comprise at least one of: bioabsorbable polymers, bioresorbable polymers, bone powder, growth factors, analgesics, antibiotics, steroids, immunosuppressants, immunostimulators, anti-inflammatory agents, osteocalcin, osteoglycin and growth factors such as bone morphogenic proteins (BMP-2, -4, -6, -7 and -9), fibroblast growth factors (FGFs), vascular endothelial growth factors (VEGFs), platelet derived growth factors (PDGFs), and transforming growth factors, antifungal agents, and antiviral agents; silver or copper micro- or nano-particles, cartilage shavings, abrasion chondroplasty, s cartilage cell allografts, stem cell autografts, s perichondral autografts, periosteal autografts, cartilage scaffolds, osteoarticular autografts, or allografts. In another aspect, the one or more surgical guides, the one or more surgical inserts, or both are manufactured to treat bone trauma, bone fractures, cancer, juvenile hallux valgus, clubfoot, congenital vertical talus, calcaneovalgus deformity, metatarsus adductus, flexible flatfoot, tarsal coalitions, or cavus foot. In another aspect, the computer-readable storage method further comprising 3-D printing one or more anatomical models of the one or more bones using the three-dimensional (3-D) image file of an ankle, a foot, or both. In another aspect, the interactive surgery planning tool can be used remotely to generate the one or more surgical guides, the one or more surgical inserts, or the one or more anatomical models of the one or more bones. In another aspect, the one or more surgical guides are configured to conform to a surface of the bones of the foot or ankle of a specific patient. In another aspect, the one or more surgical guides comprise one or more of: holes, slots, markers, pins, points, or indicia for aligning the surgical guide to a target location of the one or more bones or the foot or ankle.

As embodied and broadly described herein, an aspect of the present disclosure relates to a non-transitory computer readable medium for 3-D printing or milling one or more surgical guides, one or more surgical implants, or both, comprising instructions stored thereon, that when executed by a computer having a communications interface, one or more databases and one or more processors communicably coupled to the interface and one or more databases, perform the steps comprising: obtaining or having obtained one or more three-dimensional (3-D) image file of an ankle, a foot, or both with a medical imaging system; providing an interactive surgery planning tool that generates a surgical plan and hardware dataset comprising one or more steps in a surgical procedure of the 3-D image, one or more surgical guides, one or more implants, or both the surgical guides and implants; using the surgical dataset to generate a surgical guide dataset for one or more surgical guides, one or more surgical implants, or both; and using the surgical guide dataset to at least one of: 3-D printing or milling one or more surgical guides using the surgical guide dataset, the one or more surgical guides comprising at least one of: one or more openings for inserting a bone screw or pin; one or more slots for drilling or cutting bone; one or more slots along an edge of the one or more surgical guides for aligning the one or more surgical guides for receiving a bone screw or pin; a surface that is contoured to match a surface of a target bone; one or more indicia or predictive hole features for marking or aligning the one or more surgical guides to a bone or for marking a designation for one or more guides. In another aspect, the non-transitory computer readable medium further comprising displaying or printing a step-by-step guide for the use of the one or more surgical guides, the one or more surgical inserts, or both. In another aspect, the step-by-step guide is printed, electronic, or displayed as a 3-D rendering. In another aspect, the one or more images comprise at least one of: one or more X-rays, one or more computed tomography (CT) scans, one or more cone beam computed tomography (CBCT) scans, one or more magnetic resonance imaging (MRI) scans, an ultrasound scan, or one or more positron emission tomography (PET) scanning. In another aspect, the one or more surgical guides, the one or more surgical inserts, or both comprise at least one of: titanium, an alloy of titanium, nitinol, stainless steel, plastic, or poly ethyl ketone (PEEK). In another aspect, the one or more surgical guides, the one or more surgical inserts, or both comprise at least one of: bioabsorbable polymers, bioresorbable polymers, bone powder, growth factors, analgesics, antibiotics, steroids, immunosuppressants, immunostimulators, anti-inflammatory agents, osteocalcin, osteoglycin and growth factors such as bone morphogenic proteins (BMP-2, -4, -6, -7 and -9), fibroblast growth factors (FGFs), vascular endothelial growth factors (VEGFs), platelet derived growth factors (PDGFs), and transforming growth factors, antifungal agents, and antiviral agents; silver or copper micro- or nano-particles, cartilage shavings, abrasion chondroplasty, s cartilage cell allografts, stem cell autografts, s perichondral autografts, periosteal autografts, cartilage scaffolds, osteoarticular autografts, or allografts. In another aspect, the one or more surgical guides, the one or more surgical inserts, or both are manufactured to treat bone trauma, bone fractures, cancer, juvenile hallux valgus, clubfoot, congenital vertical talus, calcaneovalgus deformity, metatarsus adductus, flexible flatfoot, tarsal coalitions, or cavus foot. In another aspect, the non-transitory computer readable medium further comprises 3-D printing one or more anatomical models of the one or more bones using the three-dimensional (3-D) image file of an ankle, a foot, or both. In another aspect, the interactive surgery planning tool can be used remotely to generate the one or more surgical guides, the one or more surgical inserts, or the one or more anatomical models of the one or more bones. In another aspect, the one or more surgical guides are configured to conform to a surface of the bones of the foot or ankle of a specific patient. In another aspect, the one or more surgical guides comprise one or more of: holes, slots, markers, pins, points, or indicia for aligning the surgical guide to a target location of the one or more bones or the foot or ankle.

As embodied and broadly described herein, an aspect of the present disclosure relates to one or more surgical guides, surgical implants, or both, made by the methos described hereinabove.

### BRIEF DESCRIPTION OF THE DRAWINGS

For a more complete understanding of the features and advantages of the present disclosure, reference is now made to the detailed description of the disclosure along with the accompanying figures and in which:

FIG. 1 provides a summary of the subject device surgical guides including features which may be present on the device.

FIG. 2 shows a side view of a marking, positioning, and/or cutting/drilling (top) and a top view of the marking, positioning, and/or cutting/drilling (bottom).

FIG. 3 is a side view of an additive manufacturing printer for use with the present invention.

FIG. 4A shows a validation build type #1, and FIG. 4B shows a validation build type #2.

FIG. 5 is a side view of 3D metal printer.

FIG. 6 shows a first step in the method and system of the present disclosure in which a service request is initiated, in this case shown as within an application, but service request can also be made over a global telecommunications network, via a telephone call, internet, intranet, etc.

FIG. 7 shows a screenshot of the next step in the method and system of the present disclosure in which the patient data is submitted via an upload over a global telecommunications network or can be sent on a disc or medium that contains the patient image data.

FIG. 8 shows the patient images received and analyzed for review of MedCAD options.

FIG. 9 shows images of the online planning session between the MedCAD team and the surgeon. Surgeon input and approvals are an important step of the method and system of the present disclosure. By reviewing the patient's 3D anatomy with the surgeon, the method and system greatly enhances control and accuracy of the surgical planning process. Current treatments are limited to 2D X-ray images only. The AccuStride system of designers, software(s), and image data allow for unprecedented control of surgical plans, cuts, and movements.

FIG. 10 is a screen shot of a case report generated after the online planning session with the surgeon, which shows the locations for the guides on the anatomy of the patient. At this stage, it is still possible to make adjustments to the guides as requested by the surgeon or recommended by MedCAD advisors.

FIG. 11 is a screen shot that shows the part(s) list of the surgical kit that will be generated that includes the 3D guides, markings, opening(s), slots, contour, etc., that are part of the kit, including cut guide(s), the positioning of pin(s) or other indicia that will be used to guide the surgery, and any final plate(s) that may be permanently or temporarily affixed to the bone after the surgery.

FIG. 12 shows an example of guides that have been printed that include the cut slots, openings for pins; and positioning on printed bones that matches the 3D anatomy of the patient. All these components can be provided to the surgeon to confirm that the guides will properly contour to the bone, and that the surgeon will be able to properly place the guides, pins, etc., on the bone during the actual surgery.

FIG. 13 shows the results from a bunionplasty using the guides of the present disclosure.

### DETAILED DESCRIPTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

### Device Description.

The MedCAD® AccuStride® System is a novel collection of software and hardware including associated additive manufacturing equipment that provides a variety of outputs to support non-acute, non-joint replacing osteotomies in the foot and ankle. The system uses electronic medical images of the patient's anatomy obtained from the patient, provides an interface for physician input, and production of surgical guides and surgical inserts for use in surgery. The interface allows the surgeon to manipulate the original patient images for planning and executing surgery. The patient-specific outputs from the system includes anatomical models, surgical guides, and case reports. The MedCAD® AccuStride® System can be used with patients of any age. The examples herein show the use of the system for patients 12 years of age or older.

Following the MedCAD Quality System and specific Work Instructions of the present invention and using available software to manipulate 3-D medical scan images created from, e.g., Computed Tomography (CT) scans, the user is able to create patient-specific physical and digital outputs. Physical outputs include the surgical guides and surgical implants of the present invention. The process includes clinical input and review from the physician during planning and prior to delivery of the final outputs. While the process and dataflow vary somewhat based on the requirements of a given patient and physician, the following description outlines the functions of key sub-components of the system, and how they interact to produce the defined system outputs. While the system is generally operated by trained users, that is, the physician does not directly input information, with enough training any user can operate the system and then direct the printer or mill to generate the surgical guides and surgical inserts for use in surgery. The system allows for a physician to provide input for model manipulation and interactive feedback through viewing of digital models of system outputs that can be modified by an engineer or user during the planning session.

The MedCAD® AccuStride® System can be integrated into a single software system of can include two or more software modules for: (1) the design and various manufacturing equipment integrated to provide a range of anatomical models (physical and digital), (2) surgical guides, and/or (3) patient-specific case reports. The system imaging software used for the MedCAD® AccuStride® System can include Mimics (Materialise, Belgium) and GeoMagic Freeform (Artec3D, California, USA).

The MedCAD® AccuStride® System can use any input 3-D image file from medical imaging systems (i.e., CT) and/or device file. If the physician requests the system output to be compatible with a specific surgical device such as k-wires or bone clamps, the physician can specify the component and its dimensions (through literature and/or part number) or provide MedCAD with a physical sample of the device. Specific language is added to the patient-specific Case Reports to ensure the physician is aware of the critical dimension(s) of the specific output. It is at the sole discretion of the physician to select an appropriately sized surgical tool or fixation accessory to use with the specific output during surgery.

The MedCAD® AccuStride® System requires an input 3-D image file from medical imaging systems (e.g., CT) and/or device file. This input is then used, with support from the prescribing physician to provide the following potential outputs to support reconstructive surgery. Each system output is designed with physician input and reviewed by the physician prior to finalization. All outputs are used only with direct physician involvement to reduce the criticality of the outputs.

The following table identifies the device type categories and device type subcategories that define the design envelope. The below outputs along with patient-specific case reports are provided to the prescribing physician.

TABLE 1
Device type categories and device type subcategories

| Device Type Category | Device Type Subcategories |
|---|---|
| Anatomical Models | Pre-Op Model |
| | Post-Op Model |
| Surgical Guides | Marking |
| | Positioning |
| | Cutting / Drilling |

### Patient-Specific Case Reports.

MedCAD® AccuStride® System case reports provide a 2-dimensional visual representation of the patient's anatomy as it relates to surgical procedural steps derived from the physician approved treatment plan.

### Anatomical Models.

3D-printed anatomical model(s) allows the imaging data to be transformed from a two-dimensional image to a three-dimensional form (or from static images to a dynamic model), which gives a realistic impression/perspective to physicians. Unlike static planar images, the anatomical model can be viewed in any direction (0-360°) to allow for auxiliary visualization of the anatomy; therefore, it allows the physician to more effectively and comprehensively understand the specific details of the patient's anatomy. Anatomical models can be split into two distinct subcategories: Pre-Op Model and Post-Op Model. Anatomical models may come in the form of the patient's as-is anatomy (pre-operative model), or they may be a physical representation of the intended outcome as it relates to the digital treatment plan (post-operative model). Anatomical models may be sterilized and brought into the operating room; however, they are generally for information purposes only. Anatomical models are not intended to come into contact with the patient or any surgical equipment including surgical guides.

### Surgical Guides.

The surgical guides of the present invention provide for tactile placement of specific surgical procedural steps derived from the patient imaging data and the physician approved treatment plan. The guides are designed to contour the patient anatomy while transferring landmarks derived from the physician approved treatment plan to the patient anatomy.

During planning the physician may request/design guides to be produced for use during a specific step in the surgical sequence. AccuStride® System surgical guides can be split into three distinct subcategories based on their intended use: marking, positioning, and/or cutting/drilling. A single guide may be intended for multiple purposes. For example, a single guide may include features to support use in guiding instrumentation for an osteotomy and features to aid in positioning of the final anatomy following said osteotomy.

Marking guides may be used to support the marking of bony anatomy with FDA-cleared marking tools such as surgical pens/pencils to transfer the location of relevant landmarks from the treatment plan to the patient's bony anatomy. The physician can also use the marked locations on the bone as visual aid when performing cutting and/or drilling using FDA-cleared cutting and/or drilling equipment.

Positioning guides may be used to provide tactile feedback to the physician during positioning of bony anatomy as it relates to the treatment plan. Once the physician is content with the position of the bony segments, the physician may then use an FDA-cleared fixation system to fixate the bony segments together.

Cutting/drilling guides may be used to guide surgical instrumentation as required by the procedure and are compatible with commonly available FDA-cleared cutting and drilling equipment. If the physician chooses, they may temporarily secure the surgical guide with FDA-cleared k-wires and surgical bone clamps that are compatible with the device envelope. Table 2 provides Additional information on the family envelope of the subject device surgical guides.

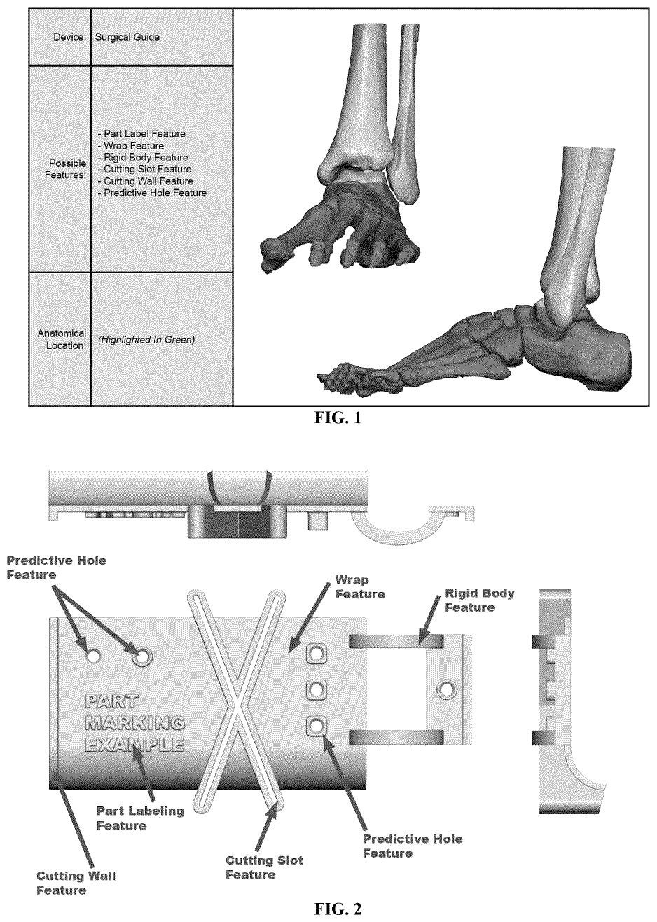

FIG. 1 provides a summary of the subject device surgical guides including features which may be present on the device. FIG. 2 shows a side view of a marking, positioning, and/or cutting/drilling (top) and a top view of the marking, positioning, and/or cutting/drilling (bottom).

TABLE 2
Surgical Guide Summary
Specification

Length
Width
Height
Degree of Curvature
Labeling Feature (Font Size)
Labeling Feature (Emboss Height)
Predictive Hole Feature (Spacing)
Predictive Hole Feature (Quantity)
Predictive Hole Feature (Inner Diameter)
Predictive Hole Feature (Proximity To Critical Anatomy)
Predictive Hole Feature (Wall Thickness Around Hole)
Predictive Hole Feature (Hole Depth To Bone)
Rigid Body Feature (Cross Section)
Cutting Slot Feature (Internal Channel Width)
Cutting Slot Feature (Internal Channel Length)
Cutting Slot Feature (Material Thickness Around Slot)
Cutting Slot Feature (Internal Channel Depth to Bone)
Cutting Wall Feature (Width)
Cutting Wall Feature (Length)
Cutting Wall Feature (Depth to Bone)
Wrap Feature (Thickness)
Fixation Hole Feature (Inner Diameter)
Fixation Hole Feature (Hole Depth To Bone)

The subject device is intended for use in patients of any age; however, the following examples are for patients 12 years of age and older. For example, and adolescent includes those patients that are greater than 12 through 21 years of age. The MedCAD® AccuStride® System is intended to be used in the adult and pediatric adolescent population. This adolescent population includes children aged 12-21 years up until but not including the 22^{nd} birthday.

The surgical instruments generated using the present invention do not impact patient growth when placed on adolescents, as it is only in use on the patient for less than 24 hours. In general, surgical treatments of hallux valgus in adolescent populations are shown to result in excellent clinical and radiological results. More specifically, osteotomies of the foot and ankle in the pediatric population allow surgeons to treat hallux valgus with minimally invasive surgery leading to less damage to important structures while reducing the risk of avascular necrosis, non-union and infection, and setting the stage for fast recovery. Use of patient specific surgical guides in the foot/ankle have been shown to produce comparable or superior surgical outcomes when compared to traditional techniques.

There are no long-term effects directly attributable to use of this device due to the limited duration of use (≤24 hours). This device is intended to be used as an aid in foot and ankle osteotomies to ensure accurate translation of the surgical plan to patient anatomy. It is known that long term complications can potentially result from incorrectly fitting surgical guides. Device accuracy has been validated through a Fit and Form Evaluation. Additionally, labeling information ensures that only CT scans of adequate quality are used in guide design and that the provided device is used prior to any anatomical changes which may impact device fit. Therefore, the use of this device does not introduce any additional risk for surgical treatments.

A summary of the available clinical literature on the prevalence and incidence of the conditions leading to use of the device can be found following this table.

In general, surgical treatments of hallux valgus in adolescent populations are shown to result in excellent clinical and radiological results. More specifically, osteotomies of the foot and ankle in the pediatric population allow surgeons to treat hallux valgus with minimally invasive surgery leading to less damage to important structures while reducing the risk of avascular necrosis, non-union and infection, and setting the stage for fast recovery. Use of patient specific surgical guides in the foot/ankle have been shown to produce comparable surgical outcomes when compared to traditional techniques.

Additional performance testing was conducted for the subject device guides which supports compatibility with the adolescent (12-21) patient population. Refer to the Performance Testing section for a full description of the test results. A summary is provided below:

### Fit and Form Evaluation.

A fit and form evaluation was performed for the subject device titanium surgical guides when used for osteotomies in the foot and ankle. This assessment evaluated the ability of the guides to product cuts that aligned with the presurgical plan when used by representative users. To summarize, comparisons were made using the digital files against the fully processed physical devices and cut anatomy by using 3D imaging. This report concluded that MedCAD can physically produce surgical accessories which properly correspond to the patient data file(s) and physician approved surgical plan and are able to accurately translate that surgical plan to the patient's anatomy.

### Biocompatibility.

The subject device UV acylate polymer anatomical models and titanium surgical guides are manufactured. ELI titanium alloy has a long-standing history as a biomaterial within orthopedic medical devices, including those with adolescent (12-21) pediatric indications. The manufacturing process for the subject device components also includes passivation as a final manufacturing step to remove all remaining residuals. As such, no further biocompatibility testing is needed for the subject device cutting/drilling guides.

The one or more openings for drilling or cutting bone; one or more indicia for marking or aligning the one or more surgical guides to a bone, one or more guides for alignment of bone, one or more inserts, or one or more instruments for use as guide(s) of the present invention can be made with, e.g., at least one of: titanium, titanium alloy, stainless steel, stainless steel alloy, aluminum, aluminum alloy, carbon fiber composite, polymeric, ceramic, composite materials, biocompatible polymer matrix reinforced with bioactive fillers and/or fibers, polyetheretherketone (PEEK), polyvinyl chloride (PVC), polyethylene polyesters, polycarbonate, Teflon coated metal, ultra-high molecular weight polyethylene (UHMWPE), urethane di methacrylate (DUDMA)/tri-ethylene glycol dimethacrylate (TEDGMA) blended resins with fillers and/or fibers nitinol, polyphenylsulfone, polycarbonate, polyetherimide, acrylic, cobalt chrome, polyamide, or acrylonitrile butadiene styrene (ABS), or combinations thereof.

Non-limiting examples of metals for use with the present invention include, e.g., aluminum, iron, titanium, steel, stainless steel, surgical stainless steel of the general alloy type of iron, carbon, chromium (12-20%) molybdenum (0.2-3%) and nickel (8-12%), martensitic steel, grade 316L austenitic steel, grade 316LVM austenitic steel, grade 316 stainless steel, cobalt chrome, or combinations thereof.

Non-limiting examples of polymers include, e.g., polyethylene, polypropylene, polyvinylalcohol, polyvinylchloride, polystyrene, polycarbonate, polyetheretherketone (PEEK), polyamide, polyacetal, polythiophene, polyethyleneoxide, polytetrafluoroethylene, an acrylic resin such as polymethylmethacrylate (PMMA), polyurethane, an epoxy resin, and polysiloxane; polylactide, polyglycolide, polycaprolactone, poly(lactide-co-glycolide), poly(lactide-co-caprolactone), poly(glycolide-co-caprolactone), polydioxanone, polytrimethylene-carbonate, poly(glycolide-co-dioxanone), polyamide-ester, polypeptide, poly-orthoesters, polymaleic acid, polyphosphazene, polyanhydride, polycebacic-anhydride, polyhydroxyalkanoate, polyhydroxybutylate, polycyanoacrylate, or combinations thereof.

Age andDegree of Physiological Maturity of the Subject.

The intended pediatric population of the subject device is the adolescent population, which includes children aged 12-21 years. The biocompatibility for this age range is similar to the adult population, and as such, the biocompatibility testing conducted is applicable to the adolescent age range as well as the adult population.

As demonstrated in the form and fit testing, the physically manufactured device was shown to match the digital output under worst-case conditions and accurately transfer the surgical plan to the patient's anatomy. Therefore, the physically manufactured device can be used with the adolescent age range.

Finally, patient growth will not have an impact on performance of the device because, generally: (1) the device is only used for less than 24 hours, and (2) the device is contraindicated in patients where significant anatomical changes have occurred between the date of manufacture of the device and the date of use. While there are no long-term effects directly attributable to use of this device due to the limited duration of use (≤24 hours), it is known that long term complications can potentially result from incorrectly fitting surgical guides leading to incorrect implementation of the intended surgical plan. For this reason, specific guidance is provided in the labeling on appropriate use of the device as it relates to device fit, which incorporates guidance specific to pediatric patients. When used in accordance with these recommendations, the MedCAD® AccuStride® System can be used safely in both the adult and adolescent population.

A shelf-life is also provided in the labeling to limit the use of the device to within 4 months of the scan date to ensure the device aligns with the anatomy. In summary, the benefits of the device outweigh any risks associated with the age and physiological maturity of the child.

### Nature and Natural History of the Clinical Condition to be Treated.

The MedCADR AccuStride® System is a collection of software and associated additive manufacturing guides that provides a variety of outputs for use in assisting preoperative planning and/or in guiding the marking of bone and/or guide surgical instruments in non-acute, non-joint replacing osteotomies in the foot and ankle for adult and pediatric patients 12 years of age and older.

There is a wide range of foot disorders and deformities in children, some of which may require operative intervention. As an example, juvenile hallux valgus (HV) is one of the most common forefoot pathologies in children and adolescents. While the symptoms occur around 10 years of age, the deformity develops much earlier. In the adult population, about 50% of patients said that their symptoms started when they were children or teenagers. Patients who have HV are more likely to be in pain than the general population and surgery is known to significantly improve quality of life. Other examples of foot disorders in children include clubfoot, congenital vertical talus, calcaneovalgus deformity, metatarsus adductus, flexible flatfoot, tarsal coalitions and cavus foot. Complicating conditions include local infection, conditions that could change the patient's anatomy during the time between obtaining the CT scan, conditions that could impact the resulting healing process, and conditions that could result from conducting the surgery.

### Safety and Effectiveness of the Device.

Appropriate biocompatibility testing was performed on the subject device guides. Every tested device went through all manufacturing steps prior to testing. Additionally, for the titanium cutting guides, a literature based toxicological risk assessment concluded that due to the combination of materials with a long history of safe use and the inclusion of passivation as a final manufacturing step to remove all remaining residuals, no further biocompatibility testing is needed for the subject device. As discussed previously, this biocompatibility aligns with use of device on the specified population. Additionally, non-clinical performance testing showed that the devices can be made to specification, accurately translate the surgical plan to the patient anatomy, and that manufactured devices do not create excessive wear as discussed previously. In summary, all testing supports that the device is safe and effective for the intended patient population.

### Likely Duration of Device Use and its Impact on Growth and Development.

Patient growth will not have an impact on performance of the device because: 1) the device is only used for less than 24 hours; and 2) the device is contraindicated in patients where significant anatomical changes have occurred between the date of manufacture of the device and the date of use. While there are no long-term effects directly attributable to use of this device due to the limited duration of use (≤24 hours), it is known that long term complications can potentially result from incorrectly fitting surgical guides leading to incorrect implementation of the intended surgical plan. For this reason, specific guidance is provided in the labeling on appropriate use of the device as it relates to device fit, which incorporates guidance specific to pediatric patients. When used in accordance with these recommendations, the MedCAD® AccuStride® System can be used safely in both the adult and adolescent population.

### Process Flow.

The MedCAD® AccuStride® System utilizes software to virtually plan clinical operations. Preoperative planning helps visualize surgery with the ability to review the case prior to entering the operating room. The process involves the use of two software packages to create the virtual digital models used to facilitate surgical planning.

The process is summarized as follows:
Data Input (CT Scan):
   Receive patient specific data such as a CT scan. MedCAD provides a scan protocol to facilitate CT Scan collection.
   Import patient scan data into medical imaging software which converts a 2D image data into 3D model output files.
Once inputs are converted to digital files for planning:
   A planning meeting between MedCAD and the physician is used to identify surgical goals.
   Treatment plan is used to develop the design of the patient specific outputs and a case report is produced for surgeon approval.
Once outputs are approved for manufacturing:
   software is used to optimize the digital output that becomes input into the additive manufacturing software.
   Additive manufacturing software is used to import digital outputs ready for production, orient the devices onto the build platform, add support material, and initiate the build.
   Physical outputs are produced via additive manufacturing
   Physical outputs are inspected for dimensional accuracy.
   Physical outputs are packaged, labeled and shipped to surgeon

### System Outputs.

The subject device produces physical and digital outputs. Physical Outputs. The physical outputs of this process are summarized in the Table 3.

TABLE 3
Physical Outputs

| | | | Patient | |
|---|---|---|---|---|
| | | Material of | Contact | Body |
| Name | Description | Construction | Duration | Contact |
| Surgical | AccuStride Surgical | Ti-6A1-4V | Limited | Tissue/ |
| Guides | Guides provide | ELI | (<24 hours) | Bone |
| | tactile placement | Titanium | | |
| | of specific | Alloy | | |
| | surgical procedural | per ASTM | | |
| | steps derived from | F136 | | |
| | the patient imaging | | | |
| | data and the surgeon | | | |
| | approved plan. | | | |
| Anatomical | AccuStride | UV curable | No contact | N/A |
| Models | Anatomical | acrylate | | |
| | Models provide a | polymers | | |
| | visual representation | | | |
| | of a specific | | | |
| | patient's | | | |
| | anatomy derived | | | |
| | from the patient | | | |
| | imaging data. | | | |
| Case | AccuStride Case | N/A | No contact | N/A |
| Reports | Reports provide | | | |
| | a visual | | | |
| | representation of | | | |
| | the patient's | | | |
| | anatomy as it | | | |
| | relates to surgical | | | |
| | procedural steps | | | |
| | derived from the | | | |
| | physician approved | | | |
| | treatment plan. | | | |

All physical outputs (excluding case reports) are manufactured via additive manufacturing. Refer to Section 11.5 for additional information on additive manufacturing information.

### Electronic Outputs.

Patient-specific case reports are supplied in PDF format and typically give guidance on:
Use of guides and surgical technique
Applicable measurements
Operative analysis

In addition to physical devices, anatomical models may be provided in STL format upon request. All digital outputs provided by MedCAD can be approved by the surgeon throughout the process.

### Manufacturing Specifications.

All system outputs are manufactured at a MedCAD facility or a MedCAD controlled contract supplier. Equipment used for additive manufacturing and post-processing is validated for its intended use prior to commercial distribution of product. Validations, maintenance, and monitoring are controlled through the MedCAD or contract manufacturers quality system, as appropriate.

Once a MedCAD® AccuStride® System output is ready for manufacturing, the build is prepared using build preparation software that can be broken into six (6) main steps:
1. Step 1: Selection of Build Material
2. Step 2: Choice of Build Placement
3. Step 3: Design of Support Material
4. Step 4: Selection of Print Parameters
5. Step 5: Creating Build Paths
6. Step 6: Post-Processing

### UV Curable Acylate Polymer Manufactured Components

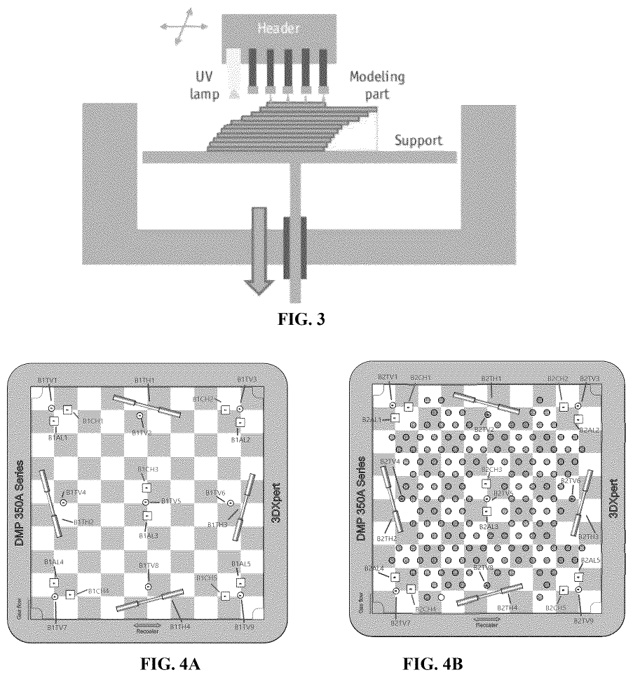

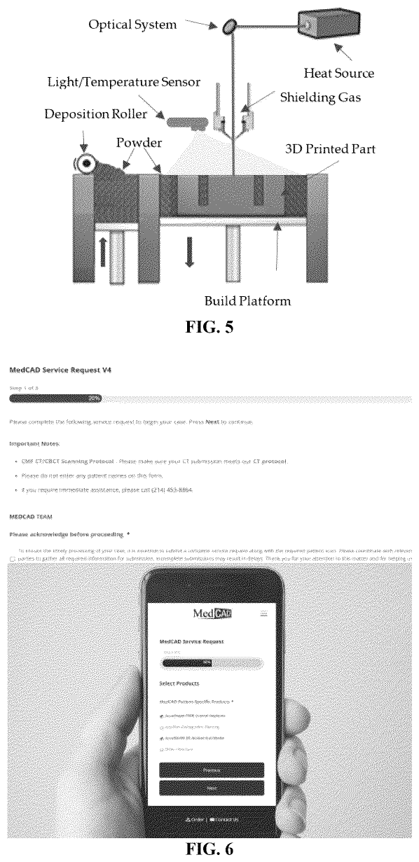

FIG. 3 is a side view of an additive manufacturing printer for use with the present invention. The subject device UV curable acrylate polymer anatomical models are manufactured using materials and methods approved for medical devices, as shown in FIG. 4. MedCAD® AccuStride® System. Printing using PolyJet 3D printing technology can be broken into 6 main steps (explained below). FIG. 4 shows device used for UV curable acrylate polymer anatomical models are manufactured using materials and methods approved for medical devices.

### Step 1: Selection of Build Material.

UV curable acrylate polymer is loaded in liquid state into the equipment and is used for the manufacturing of all subject device polymer components. A material certificate from the material manufacturer is required and inspected as part of incoming inspection for each material lot.

### Step 2: Choice of Build Placement.

When loading a device, it is oriented as flat as possible on the build platform. Each device is aligned in such a way as to minimize the height (Z direction) as much as possible. When loading a device, it may be positioned anywhere within the build tray in relation to the X and Y build directions.

### Step 3: Design of Support Material

All devices made using additive manufacturing technology are produced using a support material. Within the build preparation software, supports are generated using built in support generating software options.

### Step 4: Selection of Print Parameters

The preparation software includes print modes that vary in layer thickness and build speed. The MedCAD® AccuStride® System incorporates the use of a factory installed print mode utilizing the print mode with thickest layers and fastest printing speed.

### Step 5: Creating Build Paths

Polyjet manufacturing technology print headseject UV curable acrylate polymers in a liquid state in alignment with the 2D cross section for the specific build layer. A UV light then moves over the print bed to cure the layer, fusing the material into a solid form and adhering the printed layer to the print bed/previous layer. Support material is added as appropriate to support the manufacturing process for certain geometries (e.g., overhanging sections). This process repeats until the entire device has been manufactured from the bottom up. All physical outputs are produced solid in nature. The figure below outlines this manufacturing process.

### Step 6: Post-Processing

The bulk of the support material is then removed using various instruments. Support material is further removed using a Powerblast High Pressure Water Cleaner. Parts are then manually cleaned to remove residual support material with wire and/or nylon brushes under running tap water. The parts are inspected throughout this process to ensure removal of any remaining support material. The parts are then allowed to dry.

If requested, a polishing lathe and abrasive may be used to grind the surface of the device until layer lines are removed and a light sheen is visible. The parts are then rinsed and scrubbed and allowed to dry.

### Titanium Manufactured Components.

The subject device titanium alloy components are manufactured with the MedCAD® AccuStride® System. Printing using Direct Metal Printing (DMP) technology can be broken into 6 main steps (explained below).

### Step 1: Selection of Build Material

Ti-6Al-4V ELI titanium alloy powder complying with ASTM F136 is used for the manufacturing of all subject device titanium components and the manufacturing process complies with ASTM F3001. A material certificate from the metal powder manufacturer is inspected as part of incoming inspection for each powder lot. Finally, all powder is also passed through sieves to ensure only powder particles within the validated size range are used for manufacturing. The sieve can be anywhere from a 10, 20, 25, 30, 40, 50, 60, 63, 65, 70, 75, 80 or 90 μm sieve. Powder that does not pass through the sieve is not used.

Recycled powder may be used in the manufacture of devices. Following each build, unused powder is passed through the sieve to ensure only powder particles within the validated size range are recycled. Reusable powder (i.e., powder that passes through the sieve) will be used for the next print job; non-reusable powder (i.e., powder that does not pass through the sieve) will be discarded. Recycled and fresh powder may be mixed but all powder must be from the same lot.

### Step 2: Choice of Build Placement

When selecting an orientation for printing, the device is oriented to be as flat as possible on the build platform (i.e., minimize the height (Z direction) as much as possible). When multiple devices are present on a build platform, a minimum of 1 mm distance between devices must be preserved. Process validation testing confirms all locations in the X and Y build directions are suitable for the manufacturing of MedCAD® AccuStride® titanium guides.

Additionally, the chemistry of printed parts was analyzed. Samples were placed in the center and corners of the build plate. Aluminum, vanadium, iron, yttrium, and other trace elements were analyzed using Inductively Coupled Plasma (ICP) analysis. Carbon and sulfur were assessed through combustion, and inductively coupled plasma spectrometry was then employed. Nitrogen, hydrogen, and oxygen were analyzed via fusion. The results are summarized in the Table 4.

TABLE 4
Inductively Coupled Plasma (ICP) analysis.

| | | | | Results | | |
|---|---|---|---|---|---|---|
| | | | | Build | Build | |
| | | | | Type #1 | Type #2 | |
| | | Acceptance | | (Max % | (Max % | |
| | | Criteria | | Ob- | Ob- | |
| Element | Test Method | (%) | | served) | served) | Pass/Fail |
| Aluminum | ASTM F3001 | 5.50-6.50 | | 6.35 | 6.19 | PASS |
| Vanadium | ASTM F3001 | 3.50-4.50 | | 4 | 3.97 | PASS |
| Iron | ASTM F3001 | 0.25 | Max | 0.22 | 0.2 | PASS |
| Carbon | ASTM F3001 | 0.08 | Max | 0.02 | 0.02 | PASS |
| Nitrogen | ASTM F3001 | 0.05 | Max | 0.03 | 0.02 | PASS |
| Oxygen | ASTM F3001 | 0.13 | Max | 0.13 | 0.12 | PASS |
| Hydrogen | ASTM F3001 | 0.012 | Max | 0.0038 | 0.0025 | PASS |
| Yttrium | ASTM F3001 | 0.005 | Max | <0.005 | <0.005 | PASS |
| Other | ASTM F3001 | 0.10 | Max | <0.10 | <0.10 | PASS |
| elements, | | | | | | |
| each | | | | | | |
| Other | ASTM F3001 | 0.40 | Max | <0.40 | <0.40 | PASS |
| elements, | | | | | | |
| each | | | | | | |

Laser and print parameters are tracked on each build via the additive manufacturing equipment and flagged if any anomalies are observed. Tensile and chemistry samples/build samples are manufactured with every build.

Visual inspection and dimensional verification are performed on all build coupons and finished devices. Build coupons will be tested for the first ten (10) builds to establish a representative baseline, and following acceptable results, the subsequent frequency of build coupon testing shall be determined by the maintenance and monitoring plan. Following post-processing, printed device(s) are fit against an anatomical model created using the supplied patient-data to confirm dimensional alignment with the intended anatomical features per the physician approved surgical plan.

### Step 3: Design of Support Material

All subject devices are made using a support material as part of the manufacturing process. All support material for the subject device titanium alloy devices is also made of the same titanium alloy. Within the build preparation software, supports are automatically generated based on the design of the part to be printed. The following table outlines the support structure types that may be used, when each would be used depending on the design of the device to be printed, and how each type of support structure is removed during post-processing.

### Step 4: Selection of Print Parameters

The print preparation software automatically selects the layer thickness and printing parameters including laser power and speed based on design of the part. The subject device does not use any custom internal fill patterns or fill densities such as grids, rafts, or honeycomb-like structures. All physical outputs are produced solid in nature.

Laser and print parameters are tracked on each build via the additive manufacturing equipment and flagged if any anomalies are observed. Visual inspection and dimensional verification are performed on all build coupons and finished devices.

The additive manufacturing equipment and associated process validations are evaluated per the maintenance and monitoring plan. The additive manufacturing equipment is serviced bi-annually by technicians to ensure machine critical parameters fit within the manufacturer's published tolerances.

### Step 5: Creating Build Paths

The devices are manufactured from direct metal printing (DMP), also known as direct metal laser sintering (DMLS), using DMP equipment. In this manufacturing process, a thin layer of powder is applied to the build platform and a laser is directed to the print bed in alignment with the 2D cross section for the specific build layer, fusing the material together. The 2D cross sections and laser paths are automatically selected by the print preparation software in a manner that optimizes print time. The build platform is then lowered enough for a new layer of powder to be applied to the print bed where the process repeats until the entire device has been manufactured from the bottom up.

### Step 6: Post-Processing

Following manufacturing, the device is removed from the build platform and most of the residual powder is removed via compressed air or vacuum systems. All devices are then heat-treated. The bulk of the support material is then removed, and devices are deburred by trained users using a variety or rotary grinders/scrapers. All devices are then brushed to remove any residual particles created from the support removal process. Devices are then media blasted until all surfaces have been targeted and a uniform finish is present. Finally, the part(s) are then fit against an anatomical model to confirm dimensional alignment with the intended anatomical features per the physician approved surgical plan.

The device is then transferred to an ultrasonic cleaning tank using a detergent solution. The device is submerged within the ultrasonic tank and sonicated. The solution is then flushed, and the device is rinsed. Rinsing occurs with the device submerged in the ultrasonic tank and allowed to cavitate using DI water.

Finally, the device is passivated. The device is submerged within the ultrasonic tank and allowed to cavitate for ten (10) minutes. The solution is then flushed, and the device is rinsed. Rinsing occurs with the device in the ultrasonic tank and allowed to cavitate for twenty (20) minutes while submerged in DI water.

After the plate has been cleaned and passivated, it is allowed to dry thoroughly prior to packaging as a non-sterile packaged device.

FIG. 6 shows a first step in the method and system of the present disclosure in which a service request is initiated, in this case shown as within an application, but service request can also be made over a global telecommunications network, via a telephone call, internet, intranet, etc.

FIG. 7 shows a screenshot of the next step in the method and system of the present disclosure in which the patient data is submitted via an upload over a global telecommunications network or can be sent on a disc or medium that contains the patient image data.

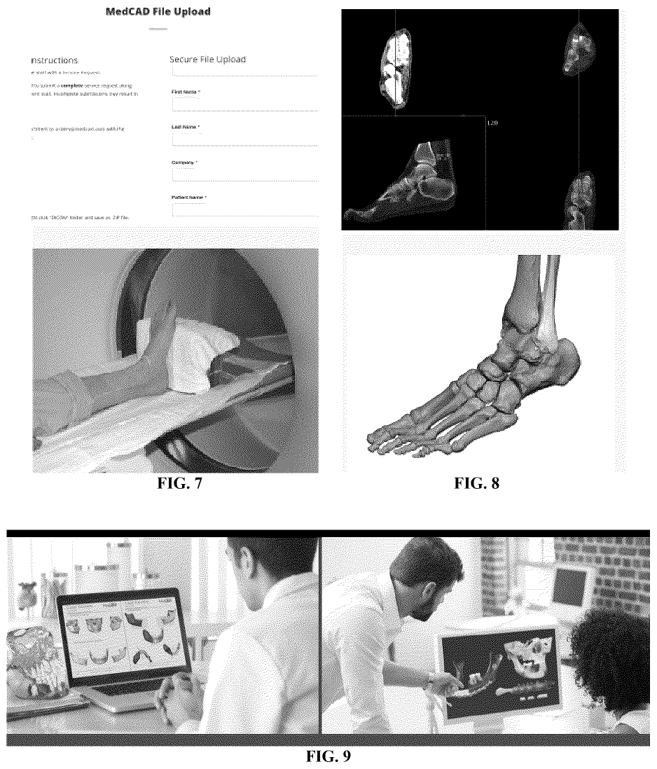

FIG. 8 shows the patient images received and analyzed for review of MedCAD options.

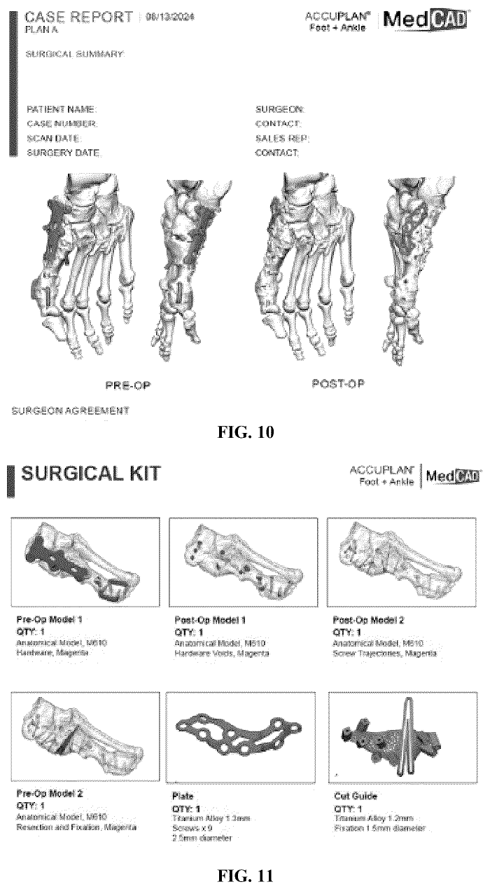

FIG. 9 shows images of the online planning session between the MedCAD team and the surgeon. Surgeon input and approvals are an important step of the method and system of the present disclosure. By reviewing the patient's 3D anatomy with the surgeon, the method and system greatly enhances control and accuracy of the surgical planning process. Current treatments are limited to 2D X-ray images only. The AccuStride system of designers, software(s), and image data allow for unprecedented control of surgical plans, cuts, and movements.

FIG. 10 is a screen shot of a case report generated after the online planning session with the surgeon, which shows the locations for the guides on the anatomy of the patient. At this stage, it is still possible to make adjustments to the guides as requested by the surgeon or recommended by MedCAD advisors.

FIG. 11 is a screen shot that shows the part(s) list of the surgical kit that will be generated that includes the 3D guides, markings, opening(s), slots, contour, etc., that are part of the kit, including cut guide(s), the positioning of pin(s) or other indicia that will be used to guide the surgery, and any final plate(s) that may be permanently or temporarily affixed to the bone after the surgery.

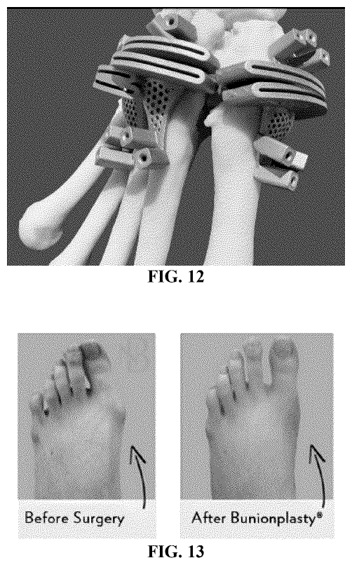

FIG. 12 shows an example of guides that have been printed that include the cut slots, openings for pins; and positioning on printed bones that matches the 3D anatomy of the patient. The anatomical models and the guides can also be provided to the surgeon to confirm that the guides will properly contour to the bone, and that the surgeon will be able to properly place the guides, pins, etc., on the bone during the actual surgery. The guides shown herein include alignment guides for inserting metal pins or wires. The increased height of the cutting slots and/or the wire or pin guide(s) will increase the precision with which the guide(s) are guiding these instruments for temporarily attaching to the bone before, during, and after the cut(s).

FIG. 13 shows the results from a bunionplasty using the guides of the present disclosure. When compared to the most experienced surgeons, which the literature summarizes as having a precision angle of 6.4+/−3.5 deg compared with freehand 10.5+/−5.5 degrees (Viehöfer, et al. (or 71.9°) for the fourth-year student, 55.5° for the third-year resident, and 70.7° for the podiatric physician (Hetherington, et al.), the present invention, even in the hands of a less experienced surgeon has a precision angle of +/−2 degrees which is a 40% improvement over the best results found in the literature. Table 5 summarizes the advantages of the system and methods of the present disclosure.

TABLE 5
Advantages of the system and methods of the present disclosure

| AccuStride Advantages | Prior Art Methods |
|---|---|
| Faster Surgeries | No Customized Guides and |
| | Implants in O.R. |
| More Control through Guidance of | No Custom-Fitted Guides for |
| Instruments | Secure Cutting |
| Drilled Hole Depth and Vector and | No Guides to Direct Customized |
| Cutting Angle Controlled and | Angles and Vectors and No |
| Known prior to Surgery | Virtual Plans Showing Vectors and |
| | Lengths |
| Customized Planning for Each | No 3D Planning Images or |
| Patient | Movement |
| 3D Printed Metal Guides and | Generic/Adjustable Guides Not |
| Plating for Strength and Fit | 3D Printed for Anatomic Fit |

A person of skill in the art would readily recognize that steps of various above-described methods can be performed by one or more programmed computers, each having one or more computer processors. Herein, some embodiments are also intended to cover program storage devices, e.g., digital data storage media, which are machine or computer-readable and encode machine-executable or computer-executable programs of instructions, wherein the instructions perform some or all of the steps of said above-described methods. The program storage devices may be, e.g., digital memories, magnetic storage media such as magnetic disks and magnetic tapes, hard drives, or optically readable digital data storage media. The embodiments are also intended to cover computers programmed to perform said steps of the above-described methods.

The functions of the various elements shown in the figures, including any functional blocks labeled as “modules”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application-specific integrated circuit (ASIC), field-programmable gate array (FPGA), read-only memory (ROM) for storing software, random access memory (RAM), and nonvolatile storage. Other hardware, conventional and/or custom, may also be included.

It is contemplated that any aspects of the disclosure discussed in this specification can be implemented with respect to any method, kit, reagent, or composition of the disclosure, and vice versa. Furthermore, compositions of the disclosure can be used to achieve methods of the disclosure.

It will be understood that particular aspects described herein are shown by way of illustration and not as limitations of the disclosure. The principal features of this disclosure can be employed in various aspects without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In aspects of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the disclosure(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any disclosure(s) in this disclosure. Neither is the “Summary” to be considered a characterization of the disclosure(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure but should not be constrained by the headings set forth herein.

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Harb, Z., Kokkinakis, M., Ismail, H., & Spence, G. (2015). Adolescent hallux valgus: A systematic review of outcomes following surgery. Journal of Children's Orthopaedics, 9 (2), 105-112.
2. Knörr, J., Soldado, F., Violas, P., Sánchez, M., Doménech, P., & de Gauzy, J. S. (2022). Treatment of hallux valgus in children and adolescents. Orthopaedics & Traumatology: Surgery & Research, 108 (1), 103168.
3. Gauci, M.-O. (2022a). Patient-specific guides in orthopedic surgery. Orthopaedics & Traumatology: Surgery & Research, 108 (1), 103154.
4. Yau, J. K., Murty, A., Kakwani, R., & Townshend, D. N. (2022). Patient specific instrumentation and total ankle arthroplasty. Foot & Ankle Orthopaedics, 7 (4).
5. Chytas, A., & Morakis, E. (2017). Foot disorders in children. Surgery (Oxford), 35(1), 48-51.
6. S, P. J. K. (n.d.). Characteristics of adolescent hallux abducto Valgus. A Retrospective Review. Journal of the American Podiatric Medical Association.
7. Wang, X., Wen, Q., Li, Y., Liu, C., Zhao, K., Zhao, H., & Liang, X. (2019). Scarf osteotomy for correction of hallux valgus deformity in adolescents. Orthopaedic Surgery, 11 (5), 873-878.
8. Cai, Y., Song, Y., He, M., He, W., Zhong, X., Wen, H., & Wei, Q. (2023). Global prevalence and incidence of HALLUX VALGUS: A systematic review and meta-analysis. Journal of Foot and Ankle Research, 16 (1).
9. Smythe, T., Rotenberg, S., & Lavy, C. (2023). The Global Birth Prevalence of Clubfoot: A Systematic Review and Meta-Analysis.
10. Zairi, M., Msakni, A., Mohseni, A. A., Othmen, A., Mensia, K., Saied, W., Bouchoucha, S., Boussetta, R., & Nessib,

M. N. (2022). Calcaneal lengthening osteotomy in the management of idiopathic flatfoot in children: Case series of twenty-One Feet. International Journal of Surgery Case Reports, 99, 107634.

What is claimed is:

1. A method of making one or more surgical guides and implants, or a one or more surgical guides and implants made by a method, comprising:
obtaining or having obtained one or more three-dimensional (3-D) image file(s) of an ankle, a foot, or both with a medical imaging system;
providing an interactive surgery planning tool that generates a surgical plan and hardware dataset comprising one or more steps in a surgical procedure of the 3-D image, one or more surgical guides, one or more implants, or both the surgical guides and implants;
using the surgical dataset to generate a surgical guide dataset for one or more surgical guides, one or more surgical implants, or both; and
using the surgical guide dataset to at least one of:
3-D printing or milling one or more surgical guides using the surgical guide dataset, the one or more surgical guides comprising at least one of:
one or more openings for inserting a bone screw or pin;
one or more slots for drilling or cutting bone;
one or more slots along an edge of the one or more surgical guides for aligning the one or more surgical guides for receiving a bone screw or pin;
a surface that is contoured to match a surface of a target bone; or
one or more indicia or predictive hole features for marking or aligning the one or more surgical guides to a bone or for marking a designation for one or more guides.

2. The method of claim, further comprising 3-D printing or milling one or more surgical inserts using the surgical guide dataset; providing a step-by-step guide for the use of the one or more surgical guides, the one or more surgical inserts, or both; or wherein the step-by-step guide is printed, electronic, or displayed as a 3-D rendering.

3. The method of claim, wherein the one or more images comprise at least one of: one or more X-rays, one or more computed tomography (CT) scans, one or more cone beam computed tomography (CBCT) scans, one or more magnetic resonance imaging (MRI) scans, an ultrasound scan, or one or more positron emission tomography (PET) scanning.

4. The method of claim, wherein at least one of:
the one or more surgical guides, the one or more surgical inserts, or both comprise at least one of: titanium, titanium alloy, stainless steel, stainless steel alloy, aluminum, aluminum alloy, carbon fiber composite, polymeric, ceramic, composite materials, biocompatible polymer matrix reinforced with bioactive fillers and/or fibers, polyetherether-ketone (PEEK), polyvinyl chloride (PVC), polyethylene polyesters, polycarbonate, Teflon coated metal, ultra high molecular weight polyethylene (UHMWPE), urethane di methacrylate (DUDMA)/tri-ethylene glycol dimethacrylate (TEDGMA) blended resins with fillers and/or fibers nitinol, polyphenylsulfone, polycarbonate, polyetherimide, polyetherketoneketone (PEKK), polyaryletherketone (PAEK), acrylic, cobalt chrome, polyamide, or acrylonitrile butadiene styrene (ABS);
the one or more surgical guides, the one or more surgical inserts, or both comprise at least one of: bioabsorbable polymers, bioresorbable polymers, bone powder, growth factors, analgesics, antibiotics, steroids, immunosuppressants, immunostimulators, anti-inflammatory agents, osteocalcin, osteoglycin and growth factors such as bone morphogenic proteins (BMP-2, -4, -6, -7 and -9), fibroblast growth factors (FGFs), vascular endothelial growth factors (VEGFs), platelet derived growth factors (PDGFs), and transforming growth factors, antifungal agents, and antiviral agents; silver or copper micro- or nano-particles, cartilage shavings, abrasion chondroplasty, s cartilage cell allografts, stem cell autografts, s perichondral autografts, periosteal autografts, cartilage scaffolds, osteoarticular autografts, or allografts;
the one or more surgical guides, the one or more surgical inserts, or both are manufactured to treat bone trauma, bone fractures, cancer, juvenile hallux valgus, clubfoot, congenital vertical talus, calcaneovalgus deformity, metatarsus adductus, flexible flatfoot, tarsal coalitions, or cavus foot;
the one or more surgical guides are configured to conform to a surface of the bones of the foot or ankle of a specific patient;
the one or more surgical guides comprise one or more of: holes, slots, markers, pins, points, or indicia for aligning the surgical guide to a target location of the one or more bones or the foot or ankle; or
the one or more surgical guides are adapted for a high tibia osteotomy, proximal phalanx and metatarsal arthroplasty, lapidus (bunion), charcot (flat foot), complex and/or revision cases in the mid and fore foot, cavus (high arch) foot correction, calcaneus fractures, or talus replacement.

5. The method of claim, further comprising 3-D printing one or more anatomical models of the one or more bones using the three-dimensional (3-D) image file of an ankle, a foot, or both.

6. The method of claim, wherein the interactive surgery planning tool can be used remotely to generate the one or more surgical guides, the one or more surgical inserts, or the one or more anatomical models of the one or more bones.

7. A system for making one or more surgical guides and implants comprising:
obtaining or having obtained one or more three-dimensional (3-D) image file of an ankle, a foot, or both with a medical imaging system;
providing an interactive surgery planning tool that generates a surgical plan and hardware dataset comprising one or more steps in a surgical procedure of the 3-D image, one or more surgical guides, one or more implants, or both the surgical guides and implants;
using the surgical dataset to generate a surgical guide dataset for one or more surgical guides, one or more surgical implants, or both; and
one or more printers or mills that following the surgical guide dataset at least one of:
3-D printing or milling one or more surgical guides using the surgical guide dataset, the one or more surgical guides comprising at least one of:
one or more openings for inserting a bone screw or pin;
one or more slots for drilling or cutting bone;
one or more slots along an edge of the one or more surgical guides for aligning the one or more surgical guides for receiving a bone screw or pin;
a surface that is contoured to match a surface of a target bone; or
one or more indicia or predictive hole features for marking or aligning the one or more surgical guides to a bone or for marking a designation for one or more guides.

8. The system of claim, further comprising 3-D printing or milling one or more surgical inserts using the surgical guide dataset; providing a step-by-step guide for the use of the one or more surgical guides, the one or more surgical inserts, or both; or wherein the step-by-step guide is printed, electronic, or displayed as a 3-D rendering.

9. The system of claim, wherein the one or more images comprise at least one of: one or more X-rays, one or more computed tomography (CT) scans, one or more cone beam computed tomography (CBCT) scans, one or more magnetic resonance imaging (MRI) scans, an ultrasound scan, or one or more positron emission tomography (PET) scanning.

10. The system of claim, wherein at least one of:
the one or more surgical guides, the one or more surgical inserts, or both comprise at least one of: titanium, titanium alloy, stainless steel, stainless steel alloy, aluminum, aluminum alloy, carbon fiber composite, polymeric, ceramic, composite materials, biocompatible polymer matrix reinforced with bioactive fillers and/or fibers, polyetherether-ketone (PEEK), polyvinyl chloride (PVC), polyethylene polyesters, polycarbonate, Teflon coated metal, ultra high molecular weight polyethylene (UHMWPE), urethane di methacrylate (DUDMA)/tri-ethylene glycol dimethacrylate (TEDGMA) blended resins with fillers and/or fibers nitinol, polyphenylsulfone, polycarbonate, polyetherimide, polyetherketoneketone (PEKK), polyaryletherketone (PAEK), acrylic, cobalt chrome, polyamide, or acrylonitrile butadiene styrene (ABS);
the one or more surgical guides, the one or more surgical inserts, or both comprise at least one of: bioabsorbable polymers, bioresorbable polymers, bone powder, growth factors, analgesics, antibiotics, steroids, immunosuppressants, immunostimulators, anti-inflammatory agents, osteocalcin, osteoglycin and growth factors such as bone morphogenic proteins (BMP-2, -4, -6, -7 and -9), fibroblast growth factors (FGFs), vascular endothelial growth factors (VEGFs), platelet derived growth factors (PDGFs), and transforming growth factors, antifungal agents, and antiviral agents; silver or copper micro- or nano-particles, cartilage shavings, abrasion chondroplasty, s cartilage cell allografts, stem cell autografts, s perichondral autografts, periosteal autografts, cartilage scaffolds, osteoarticular autografts, or allografts;
the one or more surgical guides, the one or more surgical inserts, or both are manufactured to treat bone trauma, bone fractures, cancer, juvenile hallux valgus, clubfoot, congenital vertical talus, calcaneovalgus deformity, metatarsus adductus, flexible flatfoot, tarsal coalitions, or cavus foot;
the one or more surgical guides are configured to conform to a surface of the bones of the foot or ankle of a specific patient; or
the one or more surgical guides comprise one or more of: holes, slots, markers, pins, points, or indicia for aligning the surgical guide to a target location of the one or more bones or the foot or ankle.

11. The system of claim, further comprising 3-D printing one or more anatomical models of the one or more bones using the three-dimensional (3-D) image file of an ankle, a foot, or both.

12. The system of claim, wherein the interactive surgery planning tool can be used remotely to generate the one or more surgical guides, the one or more surgical inserts, or the one or more anatomical models of the one or more bones.

13. A computer-readable storage medium comprising instructions stored thereon that, in response to execution by a processor, causes an apparatus to print or mill one or more surgical guides, one or more surgical implants, or both, comprising:
obtaining, or having obtained, and storing one or more three-dimensional (3-D) image file of an ankle, a foot, or both with a medical imaging system;
a processor comprising an interactive surgery planning tool that:
generates a surgical plan and hardware dataset comprising one or more steps in a surgical procedure of the 3-D image, one or more surgical guides, one or more implants, or both the surgical guides and implants;
using the surgical dataset to generate a surgical guide dataset for one or more surgical guides; and
3-D printing or milling one or more surgical guides using the surgical guide dataset, the one or more surgical guides comprising at least one of:
one or more openings for inserting a bone screw or pin;
one or more slots for drilling or cutting bone;
one or more slots along an edge of the one or more surgical guides for aligning the one or more surgical guides for receiving a bone screw or pin;
a surface that is contoured to match a surface of a target bone; or
one or more indicia or predictive hole features for marking or aligning the one or more surgical guides to a bone or for marking a designation for one or more guides.

14. The computer-readable storage medium of claim, further comprising providing a step-by-step guide for the use of the one or more surgical guides, the one or more surgical inserts, or both, wherein the step-by-step guide is printed, electronic, or displayed as a 3-D rendering.

15. The computer-readable storage medium of claim, wherein the one or more images comprise at least one of: one or more X-rays, one or more computed tomography (CT) scans, one or more cone beam computed tomography (CBCT) scans, one or more magnetic resonance imaging (MRI) scans, an ultrasound scan, or one or more positron emission tomography (PET) scanning.

16. The computer-readable storage medium of claim, wherein at least one of:
the one or more surgical guides, the one or more surgical inserts, or both comprise at least one of: titanium, an alloy of titanium, nitinol, stainless steel, plastic, or poly ethyl ketone (PEEK);
the one or more surgical guides, the one or more surgical inserts, or both comprise at least one of: bioabsorbable polymers, bioresorbable polymers, bone powder, growth factors, analgesics, antibiotics, steroids, immunosuppressants, immunostimulators, anti-inflammatory agents, osteocalcin, osteoglycin and growth factors such as bone morphogenic proteins (BMP-2, -4, -6, -7 and -9), fibroblast growth factors (FGFs), vascular endothelial growth factors (VEGFs), platelet derived growth factors (PDGFs), and transforming growth factors, antifungal agents, and antiviral agents; silver or copper micro- or nano-particles, cartilage shavings, abrasion chondroplasty, s cartilage cell allografts, stem cell autografts, s perichondral autografts, periosteal autografts, cartilage scaffolds, osteoarticular autografts, or allografts;
the one or more surgical guides, the one or more surgical inserts, or both are manufactured to treat bone trauma, bone fractures, cancer, juvenile hallux valgus, clubfoot, congenital vertical talus, calcaneovalgus deformity, metatarsus adductus, flexible flatfoot, tarsal coalitions, or cavus foot;
the one or more surgical guides are configured to conform to a surface of the bones of the foot or ankle of a specific patient; or
the one or more surgical guides comprise one or more of: holes, slots, markers, pins, points, or indicia for aligning the surgical guide to a target location of the one or more bones or the foot or ankle.

17. The computer-readable storage method of claim, further comprising 3-D printing one or more anatomical models of the one or more bones using the three-dimensional (3-D) image file of an ankle, a foot, or both.

18. The computer-readable storage method of claim, wherein the interactive surgery planning tool can be used remotely to generate the one or more surgical guides, the one or more surgical inserts, or the one or more anatomical models of the one or more bones.

19. A non-transitory computer readable medium for 3-D printing or milling one or more surgical guides, one or more surgical implants, or both, comprising instructions stored thereon, that when executed by a computer having a communications interface, one or more databases and one or more processors communicably coupled to the interface and one or more databases, perform the steps comprising:
obtaining or having obtained one or more three-dimensional (3-D) image file of an ankle, a foot, or both with a medical imaging system;
providing an interactive surgery planning tool that generates a surgical plan and hardware dataset comprising one or more steps in a surgical procedure of the 3-D image, one or more surgical guides, one or more implants, or both the surgical guides and implants;
using the surgical dataset to generate a surgical guide dataset for one or more surgical guides, one or more surgical implants, or both; and
using the surgical guide dataset to at least one of:
3-D printing or milling one or more surgical guides using the surgical guide dataset, the one or more surgical guides comprising at least one of:
one or more openings for inserting a bone screw or pin;
one or more slots for drilling or cutting bone;
one or more slots along an edge of the one or more surgical guides for aligning the one or more surgical guides for receiving a bone screw or pin;
a surface that is contoured to match a surface of a target bone; or
one or more indicia or predictive hole features for marking or aligning the one or more surgical guides to a bone or for marking a designation for one or more guides.

20. The non-transitory computer readable medium of claim, further comprising providing a step-by-step guide for the use of the one or more surgical guides, the one or more surgical inserts, or both; or wherein the step-by-step guide is printed, electronic, or displayed as a 3-D rendering.

21. The non-transitory computer readable medium of claim, wherein the one or more images comprise at least one of: one or more X-rays, one or more computed tomography (CT) scans, one or more cone beam computed tomography (CBCT) scans, one or more magnetic resonance imaging (MRI) scans, an ultrasound scan, or one or more positron emission tomography (PET) scanning.

22. The non-transitory computer readable medium of claim, wherein at least one of:
the one or more surgical guides, the one or more surgical inserts, or both comprise at least one of: titanium, an alloy of titanium, nitinol, stainless steel, plastic, or poly ethyl ketone (PEEK);
the one or more surgical guides, the one or more surgical inserts, or both comprise at least one of: bioabsorbable polymers, bioresorbable polymers, bone powder, growth factors, analgesics, antibiotics, steroids, immunosuppressants, immunostimulators, anti-inflammatory agents, osteocalcin, osteoglycin and growth factors such as bone morphogenic proteins (BMP-2, -4, -6, -7 and -9), fibroblast growth factors (FGFs), vascular endothelial growth factors (VEGFs), platelet derived growth factors (PDGFs), and transforming growth factors, antifungal agents, and antiviral agents; silver or copper micro- or nano-particles, cartilage shavings, abrasion chondroplasty, s cartilage cell allografts, stem cell autografts, s perichondral autografts, periosteal autografts, cartilage scaffolds, osteoarticular autografts, or allografts;
the one or more surgical guides, the one or more surgical inserts, or both are manufactured to treat bone trauma, bone fractures, cancer, juvenile hallux valgus, clubfoot, congenital vertical talus, calcaneovalgus deformity, metatarsus adductus, flexible flatfoot, tarsal coalitions, or cavus foot;
the one or more surgical guides are configured to conform to a surface of the bones of the foot or ankle of a specific patient; or
the one or more surgical guides comprise one or more of: holes, slots, markers, pins, points, or indicia for aligning the surgical guide to a target location of the one or more bones or the foot or ankle.

23. The non-transitory computer readable medium of claim, further comprising 3-D printing one or more anatomical models of the one or more bones using the three-dimensional (3-D) image file of an ankle, a foot, or both.

24. The non-transitory computer readable medium of claim, wherein the interactive surgery planning tool can be used remotely to generate the one or more surgical guides, the one or more surgical inserts, or the one or more anatomical models of the one or more bones.
